(19) United States
(12) Patent Application Publication
(10) Pub. No.: US 2025/0325782 A1
(43) Pub. Date: Oct. 23, 2025

(54) Systems and Methods for Diagnosis and Treatment of Small Patent Urachal Duct Remnant (SPUDR)

(71) Applicant: Barrie Steed, Calgary (CA)

(72) Inventor: Barrie Steed, Calgary (CA)

(21) Appl. No.: 19/175,487

(22) Filed: Apr. 10, 2025

Related U.S. Application Data
(60) Provisional application No. 63/637,678, filed on Apr. 23, 2024.

(51) Int. Cl. A61M 25/00; A61M 25/01; A61M 25/06; A61M 25/09

(52) U.S. Cl. CPC A61M 25/0021; A61M 25/0662; A61M 25/09; A61M 2025/0042; A61M 2025/0175; A61M 2025/0681; A61M 2202/0007; A61M 2202/04; A61M 2210/1085; A61M 2210/1089

(57) ABSTRACT

Systems and methods for treatment of small patent urachal duct remnant (SPUDR) are described. The systems include kits for treatment of SPUDR, the kits including a microwire, a microcatheter, a support catheter and a sheath within common packaging. The kit may include a sclerosing agent or glue as well as instructions for treatment of SPUDR. Methods for treatment may include steps of navigating an assembly of a microwire and microcatheter to the SPUDR and injecting a sclerosing agent or glue into an orifice of the SPUDR.

### CROSS-REFERENCE TO RELATED APPLICATION

This application is a U.S. Non-Provisional Application that claims priority to U.S. Provisional Patent Application No. 63/637,678, filed on Apr. 23, 2024, that is hereby incorporated by reference in its entirety.

### FIELD

Systems and methods for treatment of small patent urachal duct remnant (SPUDR) are described. The systems include kits for treatment of SPUDR, the kits including a microwire, a microcatheter, a support catheter and a sheath within common packaging. The kit may include a sclerosing agent or glue as well as instructions for treatment of SPUDR. Methods for treatment may include steps of navigating an assembly of a microwire and microcatheter to the SPUDR and injecting a sclerosing agent or glue into an orifice of the SPUDR.

### BACKGROUND

Lower urinary tract symptoms entail a large differential diagnosis. While urinary tract infections with positive bacterial cultures are often easily categorized, chronic symptoms are difficult to manage and have a variety of proposed mechanisms.

For example, there is a large population of both adult and pediatric patients that have persisting or recurrent symptoms of bladder infection or interstitial cystitis/bladder pain with the cause unknown. These symptoms may be manifested as intermittent bed wetting, urgency, frequency and painful voiding and may present without any associated proven bladder infection.

These patients may exhibit symptoms typical of bladder infections but with a negative bacterial culture. Such patients are often treated with antibiotics but may poorly respond. The exact mechanism of persistent and recurrent bladder infection (BI) and interstitial cystitis (IC) is poorly understood, and management provides moderate relief.

In those cases of known infection, BI and IC is commonly caused by strains of E coli invading the uroepithlium, resulting in intracellular bacterial communities (IBCs).

In view of the foregoing, there is a need for improved diagnosis and treatment of persistent and recurrent bladder infection (BI) and interstitial cystitis (IC).

### SUMMARY

In accordance with the disclosure, there is provided a kit for treatment of a small patent urachal duct remnant (SPUDR) including: a microwire, a microcatheter, a support catheter and a sheath within common packaging, each of the microwire, microcatheter, support catheter and sheath having an outside diameter (OD) and internal diameter (ID) enabling telescopic assembly of the support catheter within the sheath, the microcatheter within the support catheter and the microwire within the microcatheter, wherein the sheath has an OD for insertion within the urethra of a patient and a length to extend from a urethral opening to the bladder, the support catheter having a length longer than the sheath to extend from the urethral opening to the bladder, the microcatheter having a length longer than the support catheter to extend from the urethral opening to the SPUDR, and the microwire having a length longer than the microcatheter to extend from the urethral opening to the SPUDR.

In various embodiments:
The kit includes instructions for treatment of SPUDR.
The kit includes a syringe configured to connect to the microcatheter for delivery of a treatment fluid through the microcatheter.
The kit includes a treatment fluid.
The treatment fluid is a sclerosing agent.
The treatment fluid is a glue.

In another aspect, a method of treating small patent urachal duct remnant (SPUDR) is described, the method including the steps of: identifying a SPUDR via ultrasound imaging of a patient; upon identification of a SPUDR, introducing a catheter system as described into the bladder of a patient; steering the microwire and microcatheter to an orifice of the SPUDR and inserting the microcatheter into the orifice; withdrawing the microwire from the microcatheter; injecting a treatment fluid into the microcatheter wherein the treatment fluid is injected into the orifice; and, withdrawing the catheter system from the bladder.

In various embodiments:
The step of injecting includes injecting a sclerosing fluid.
The step of injecting includes injecting a glue.

### BRIEF DESCRIPTION OF THE DRAWINGS

Various objects, features and advantages of the disclosure will be apparent from the following description of particular embodiments, as illustrated in the accompanying drawings. The drawings are not necessarily to scale, emphasis instead being placed upon illustrating the principles of various embodiments of the disclosure. Similar reference numerals indicate similar components.

FIG. 1 is an ultrasound image of a small patent urachal duct remnant (SPUDR).

FIG. 1A is an ultrasound image of a SPUDR showing a tract.

FIG. 2 is a sketch of the bladder and a SPUDR.

FIG. 3 is a sketch of the bladder and a first method of removal of a SPUDR via intra-bladder catheters in accordance with a method of the disclosure.

FIG. 3A is an ultrasound image showing an intra-bladder catheter treatment method utilizing catheters in accordance with one method of the disclosure.

FIG. 3B is a sketch of a treatment method utilizing an ablative material or glue in accordance with different methods of the disclosure.

FIG. 4 is a sketch of a treatment method via transdermal access in accordance with an alternative method of the disclosure.

FIG. 4A is an ultrasound image showing a transdermal treatment method in accordance with one method of the disclosure.

FIG. 5 is a sketch of a catheter system kit in accordance with one embodiment of the disclosure.

### DETAILED DESCRIPTION

With reference to the figures, systems and methods of diagnosing and treating persistent and recurrent bladder infection (BI) and interstitial cystitis (IC) are described.

### Terminology

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Spatially relative terms may be used herein for ease of description to describe one element or feature's relationship to another element(s) or feature(s) as illustrated in the figures. It will be understood that the spatially relative terms are intended to encompass different orientations of described features in use or operation in addition to the orientation depicted in the figures. For example, terms such as “upwardly”, “downwardly”, “vertical”, “horizontal”, “proximal” and “distal” and the like are used herein for the purpose of general explanation only unless specifically indicated otherwise.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present.

It will be understood that, although the terms “first”, “second”, etc. may be used herein to describe various elements, components, etc., these elements, components, etc. should not be limited by these terms. These terms are only used to distinguish one element, component, etc. from another element, component. Thus, a “first” element, or component discussed herein could also be termed a “second” element or component without departing from the teachings of the present disclosure. In addition, the sequence of operations (or steps) is not limited to the order presented in the claims or figures unless specifically indicated otherwise.

Other than described herein, or unless otherwise expressly specified, all of the numerical ranges, amounts, values, ratios, etc. may be read as if prefaced by the word “about” even though the term “about” may not expressly appear with the value, amount, or range. Accordingly, unless indicated to the contrary, the numerical parameters set forth in the description are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques. Distances may be referenced as “anatomical distances” which are construed to mean typical/average distances between anatomical features in human patient populations as understood by those skilled in the art.

Unless otherwise defined, all technical and scientific terms used herein have the same meaning as commonly understood by one of ordinary skill in the art.

Various aspects of the disclosure will now be described with reference to the figures. The disclosure may, however, be embodied in many different forms and should not be construed as limited to the embodiments set forth herein; rather, these embodiments are provided so that this disclosure will be thorough and complete, and will fully convey the scope of the disclosure to those skilled in the art. Moreover, the drawings are not necessarily drawn to scale and are intended to emphasize principles of operation rather than precise dimensions.

### Rationale

The inventor, who is experienced in the diagnosis and treatment of bladder conditions, has determined that a small patent urachal duct remnant (SPUDR) exists in many patients and is a cause of persistent and recurrent bladder infection (BI) and interstitial cystitis (IC) that may present as symptoms of bladder inflammation without another known cause.

By way of background and hypothesis, persistent and recurrent bladder infection (BI) and interstitial cystitis (IC) requires an internal source for harboring bacteria, capable of reseeding the bladder wall that thereafter results in symptoms of bladder inflammation.

For example, if dormant bacteria is activated and experiences exponential growth within the bladder, virulent toxin(s) of the growing bacteria dissipates quickly, often resulting in acute BI. In addition, quiescent toxin often declines slowly, resulting in low grade foci of inflammation over longer time frames causing IC. Confusion about the cause of IC can occur when quiescent and virulent strains of bacteria activate separately or at the same time.

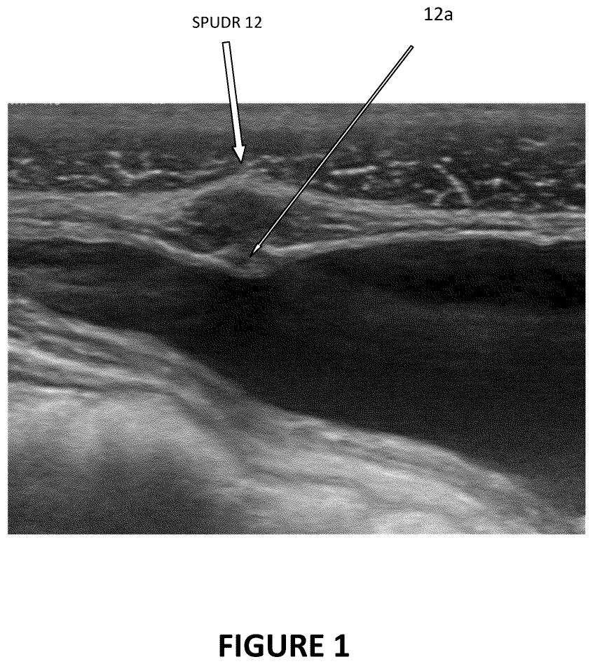

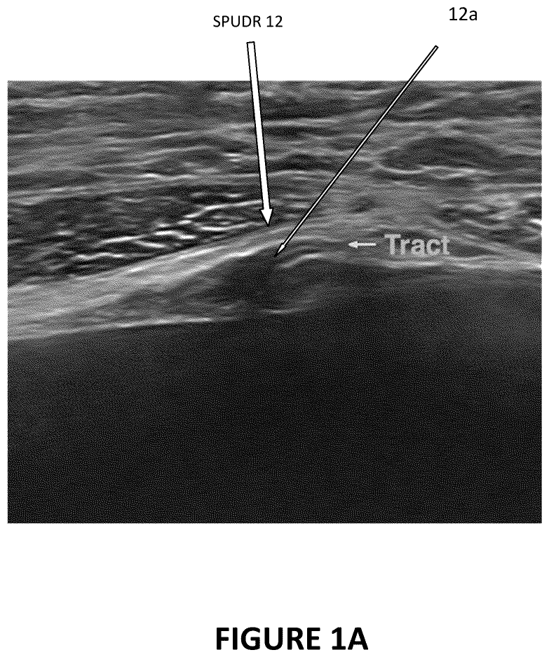

Importantly, the inventor has identified via imaging that many patients developing such symptoms have a small remnant structure on the bladder wall, identifiable as a small orifice with sufficient size that bacteria may be contained within the orifice, remain dormant for periods of time and periodically flourish before returning to dormancy. This structure is referred to herein as a SPUDR (FIGS. 1 and 1A).

The SPUDR has dimensions and an opening or tract that may prevent urine from flushing the bacteria from the structure such that bacteria may remain dormant within the SPUDR. At various times, as a result of various triggers, these harbored bacteria may enter lag and exponential growth phases, that thereafter result in symptoms. After a period of time, the bacteria return to a dormant state until a subsequent trigger occurs. It is speculated that E coli can survive in the tract for variable time frames (up to years), which entails a form of cocooning and/or hibernation, wherein they can attach to the SPUDR wall and not easily washed out.

### Description of a SPUDR on Ultrasound Imaging

As observed via imaging (see FIGS. 1 and 1A), a SPUDR 12 is a single homogeneous, hypodense to mildly echogenic soft tissue mass, blending in and confined to the area of the anterior middle bladder wall. The mass leaves the bladder mucosa undisturbed, is wider than taller, and is typically 1 to 2.5 cm in greatest dimension.

The SPUDR mass may also show a centrally placed round tubular tract filled with clear fluid, having a blind ending superiorly, at 1 to 3 mm in greatest diameter, often seen as two thin parallel lines. Important to patency is detection of an orifice, at 1 to 3 mm, seen as a round echo lucent defect in the bladder wall mucosa, connecting bladder fluid to the remnant tubular tract. The orifice confirms patency with the bladder lumen. The natural course of the tract is superior through the mass, forming an acute angle at the mucosal orifice, making visualization of the orifice unlikely at cystoscopy.

### Scanning Technique for Visualization

An ultrasound machine can be used for visualization of the SPUDR (e.g. a Siemens 2000 ultrasound machine). Important to visualization is an optimal degree of bladder distension. A SPUDR is not seen well with bladder wall over-distension.

If SPUDR is not seen, the patient can void for a slow count to 10, stop and be re-examined, until good visualization is achieved. The bladder could be underfilled and requires waiting with re-examination after an interval of time. Repetition of this voiding pattern visualizes a SPUDR mass and tube in suspected cases. Pediatric populations may require refilling and repeat examination, as compliance can be difficult.

Gross bladder abnormalities can be excluded with a curved ultrasound probe.

The following steps may typically be conducted and various factors considered:
a. Utilizing a quality high frequency linear probe, scan the superior, anterior, midline of the distended bladder wall for a soft tissue mass, in saggital, transverse and oblique views.
b. If a SPUDR is suspected, the soft tissue mass is mildly hypodense to echogenic, in the range of 1 to 2.5 cm.
c. Scan the mass to detect a small round tubular fluid filled structure, often seen as two linear parallel lines having a tract like appearance, 1 to 2 mm in diameter.
d. Clear fluid in the tract supports the tract is patent.
e. To identify the orifice, follow the tract toward the mucosa to detect a round hypodense mucosal orifice of 1 to 2 mm to confirm patency of the SPUDR.
f. If a tubular tract is not seen, examine the mucosa in the central area of the mass to detect the 1 to 2 mm orifice defect in the mucosa. If found, a patent tract is often seen passing superior to the orifice.
g. A patent orifice and fluid filled tract is usually found in the mass with a fastidious ultrasound examination, with the right clinical profile.
h. If the SPIDUR is not seen, have the patient adjust bladder pressure.

Other Imaging modalities can be used to visualize a SPUDR. For example, a CT with contrast in the bladder may be conducted. Typically, a CT diagnosis, as above, would require optimal bladder wall tension, in order to fully fill and demonstrate the tract. At cystoscopy the tract is at a more acute angle to the orifice and has never been recorded as seen blending in with normal mucosal tissues.

### Theory of Action and Past Diagnosis/Treatment

Without being limited or bound to specific theory, it is postulated random bacteria in the bladder lumen can be voided, attach to the wall causing bladder infection and/or enter the SPUDR tract. E coli or other bacteria harboring in the tract may not aggressively attach to the SPUDR tract wall, with no evidence of inflammation in the SPUDR tract or tissue mass. However, E coli could attach to the tract wall for purposes of quiescent hibernation up to 6 months or more. If infected, effects can be minimized by drainage through the patent orifice.

Mild symptoms in the pediatric population are often treated initially with antibiotics, even with a negative culture. When these symptoms persist, the symptomatology is often labeled psychological or behavioral. Children may be protected from more intense symptoms by the ability to more completely void, leaving less E. coli to inflame the bladder wall. This also may contribute to the higher probability of a negative bacterial culture. Adults have a larger post-void residual acting as a reservoir for E. coli and bacteria, resulting in positive C&S with more intense symptoms than in pediatrics.

### Systems and Methods for SPUDR Treatment

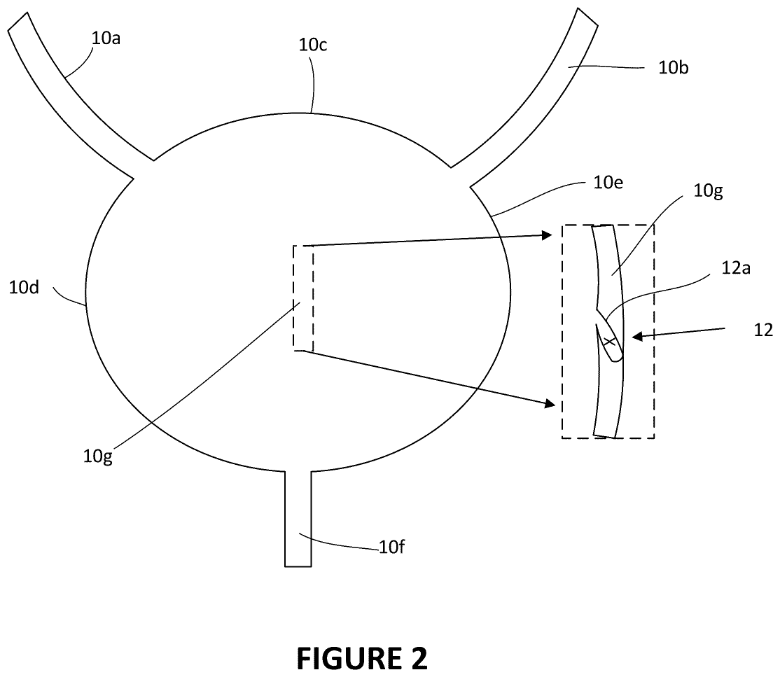

As shown schematically in FIG. 2, the bladder 10 is shown having ureters 10a, 10b, dome 10c, right lateral wall 10d, left lateral wall 10e, anterior wall 10g and urethra 10f.

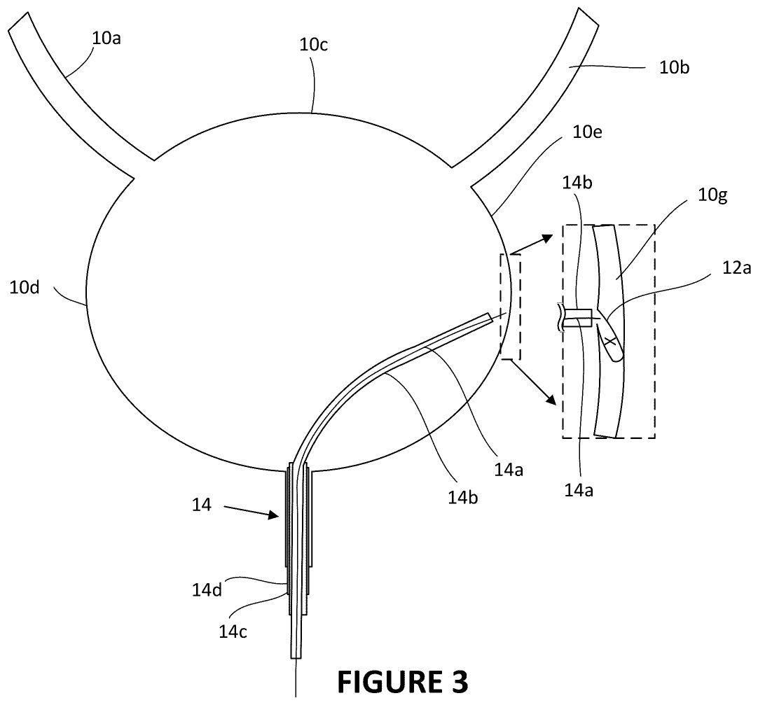

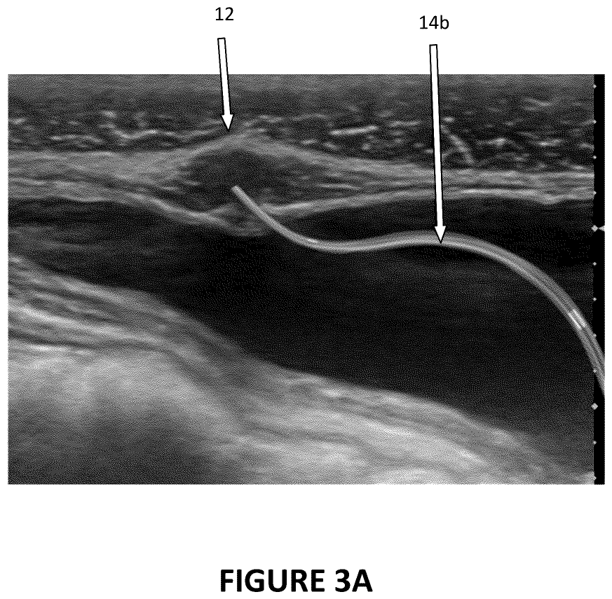

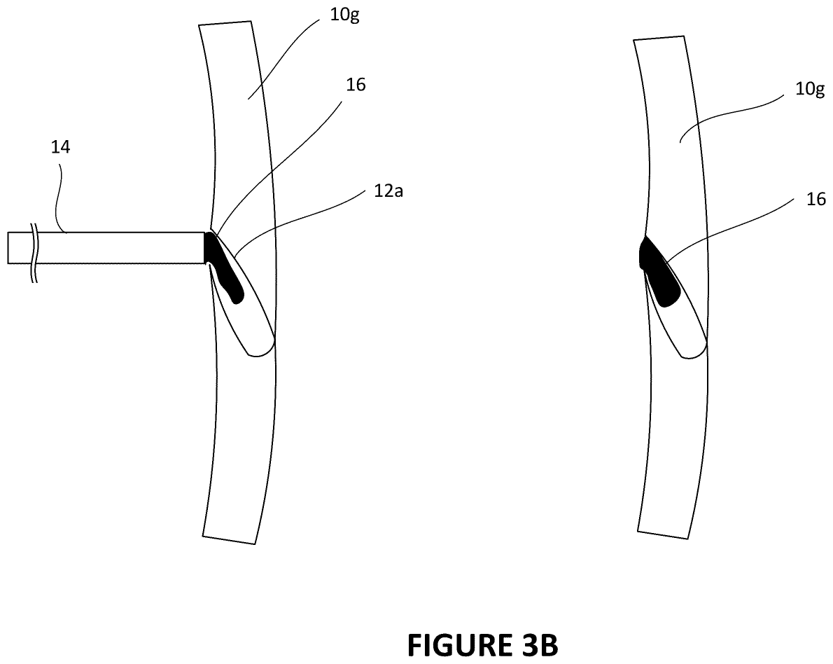

As described above, a SPUDR 12 (see expanded region) may exist in the anterior wall 10g of the bladder (shown schematically as a small section of the bladder wall in FIGS. 2 and 3) as a small orifice 12a that may harbor bacteria X.

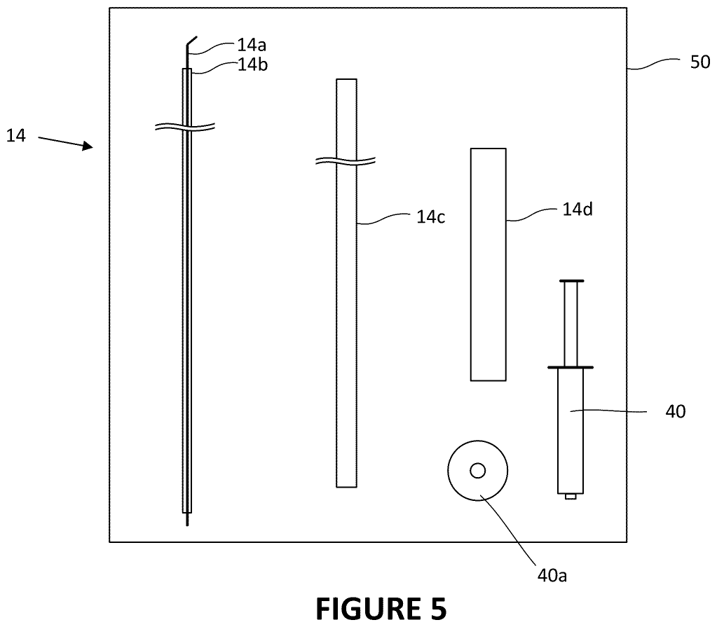

In accordance with a first embodiment, and as shown in FIGS. 3, 3A and 5, a catheter system 14, including a microwire 14a, microcatheter 14b, guide catheter 14c and sheath 14d system is described that may be navigated under ultrasound/fluoroscopy guidance to the SPUDR via the urethra.

As shown, the catheter system is a co-axial assembly of catheters that enables navigation of the microcatheter to the SPUDR and enables treatment.

Generally, the microwire and microcatheter can be steered by the interventionist to the SPUDR using ultrasound/fluoroscopy imaging wherein the support catheter provides stiffness/support to the system to enable fine guidance of the microwire and microcatheter through selective co-axial movements of the microwire and microcatheter. The urethra is typically protected by the sheath 14c, which minimizes frictional contact of the system within the urethra. The sheath protects against bladder infection, as the urethra can support bacteria for a short segment in the distal end of the urethra.

The system enables fluid to be introduced and removed from the bladder via a fluid pump (e.g. a syringe connected to the support catheter and/or sheath) to enable the interventionist to adjust the pressure within the bladder to enable SPUDR identification and, hence, navigation.

As shown in FIG. 3B (schematic image) when positioned over the opening 12a, an agent 16 may be injected into the SPUDR orifice from the catheter to sterilize and/or ablate the SPUDR resulting in occlusion and/or elimination of the orifice. Suitable agents may be sclerosing agents such as alcohol (e.g. >80% w/v) or glues such as Onyx™. Pure ethanol may be effective.

The microcatheter is typically about 12-18 inches in length having an OD of about 1-3 French and an ID to enable passage of the microwire and the treatment agent and, otherwise having a diameter enabling positioning within the duct.

The microwire is typically about 15-24 inches in length and may have a deformable tip to enable torquing/steering of the microcatheter. The microwire has an OD to enable passage within the microcatheter.

The guide/support catheter may be about 8-12 inches in length having an ID to enable passage of the microcatheter and an OD to slide within the sheath.

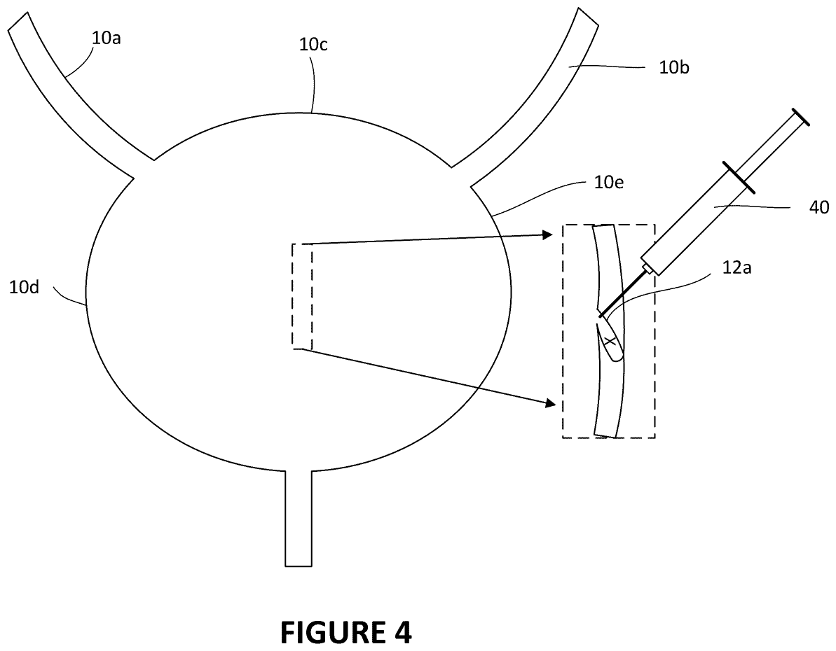

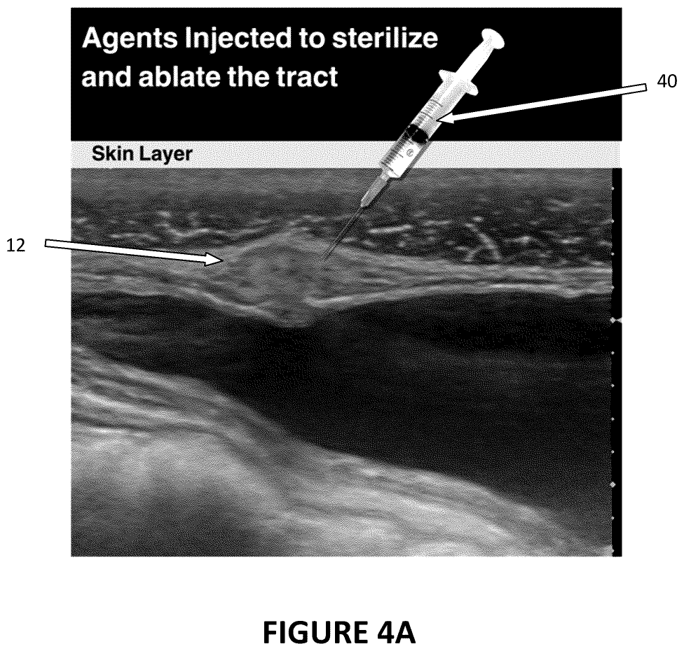

FIGS. 4 and 4A show an alternate transdermal treatment method where a syringe 40 containing a sclerosing agent or glue is directed to the SPUDR under ultrasound guidance as described above.

FIG. 5 is a sketch showing the catheter system 14 as a kit within packaging 50. As shown, the kit may include the microwire 14a, microcatheter 14b, support catheter 14c and sheath 14d within single packaging enabling an interventionist to readily access the equipment to effect treatment. The kit may include a syringe 40 and vial of sclerosing agent or glue 40a. The microwire and microcatheter may be pre-assembled and the kit may include instructions for use and treatment of SPUDR.

Although the present disclosure has been described and illustrated with respect to preferred embodiments and preferred uses thereof, it is not to be so limited since modifications and changes can be made therein which are within the full, intended scope of the disclosure as understood by those skilled in the art.

1. A kit for treatment of a small patent urachal duct remnant (SPUDR) comprising:
a microwire, a microcatheter, a support catheter and a sheath within common packaging, each of the microwire, microcatheter, support catheter and sheath having an outside diameter (OD) and internal diameter (ID) enabling telescopic assembly of the support catheter within the sheath, the microcatheter within the support catheter and the microwire within the microcatheter, wherein the sheath has an OD for insertion within the urethra of a patient and a length to extend from a urethral opening to the bladder, the support catheter having a length longer than the sheath to extend from the urethral opening to the bladder, the microcatheter having a length longer than the support catheter to extend from the urethral opening to the SPUDR, and the microwire having a length longer than the microcatheter to extend from the urethral opening to the SPUDR.

2. The kit as in claim 1 further comprising instructions for treatment of SPUDR.

3. The kit as in claim 1 further comprising a syringe configured to connect to the microcatheter for delivery of a treatment fluid through the microcatheter.

4. The kit as in claim 3 further comprising a treatment fluid.

5. The kit as in claim 4 where the treatment fluid is a sclerosing agent.

6. The kit as in claim 4 where the treatment fluid is a glue.

7. A method of treating small patent urachal duct remnant (SPUDR) comprising the steps of:
identifying a SPUDR via ultrasound imaging of a patient;
upon identification of a SPUDR, introducing a catheter system as in claim 1 into the bladder of a patient;
steering the microwire and microcatheter to an orifice of the SPUDR and inserting the microcatheter into the orifice;
withdrawing the microwire from the microcatheter;
injecting a treatment fluid into the microcatheter wherein the treatment fluid is injected into the orifice; and,
withdrawing the catheter system from the bladder.

8. The method as in claim 7 wherein the step of injecting includes injecting a sclerosing fluid.

9. The method as in claim 7 wherein the step of injecting includes injecting a glue.
